(19) United States
(12) Patent Application Publication
(10) Pub. No.: US 2017/0369455 A1
(43) Pub. Date: Dec. 28, 2017

(54) COMBINATION OF UV ABSORBER AND PIGMENT FOR PROTECTION OF SUBSTRATES FROM UV/VIS-RADIATION

(71) Applicant: BASF SE, Ludwigshafen (DE)

(72) Inventors: Rolf ALLES, Hessheim (DE); Katharina FRITZSCHE, Weil am Rhein (DE); Wolfgang PETER, Altlussheim (DE); Adalbert BRAIG, Binzen (DE)

(73) Assignee: BASF SE, Ludwigshafen (DE)

(21) Appl. No.: 15/535,945

(22) PCT Filed: Dec. 18, 2015
(86) PCT No.: PCT/EP2015/080533

§ 371 Date: Jun. 14, 2017

(30) Foreign Application Priority Data
Dec. 19, 2014 (EP) 14199330.3

(51) Int. Cl. C07D 251/24; C09D 7/12; C09D 5/32; C09K 15/30; C08K 5/3492

(52) U.S. Cl. CPC C07D 251/24; C09K 15/30; C08K 5/3492; C09D 5/32; C09D 7/1241

(57) ABSTRACT

A coating composition, which contains one or more compounds (A) according to formula (I) and a pigment (B) having a minimum integrated transmittance within the range of 380 to 600 nm, is provided:

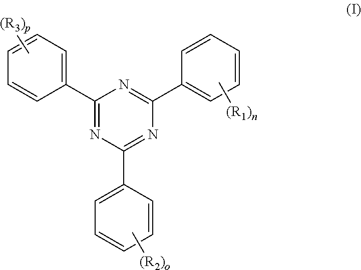

In formula (I), each R₁, R₂ and R₃ is independently —OR₄, R₄ being independently hydrogen or a C₁ to C₂₄ hydrocarbyl group optionally containing heteroatoms, or C₁ to C₂₄ hydrocarbyl groups optionally containing heteroatoms; n is 2, 3, 4 or 5; o is 2, 3, 4 or 5; p is 2, 3, 4 or 5; with the proviso that at least one of R₁ and at least one of R₂ and at least one of R₃ is —OR₄. A coating is obtained by applying the coating composition to a substrate for protecting the substrate against UV/Vis-radiation or for stabilizing the substrate against the deleterious influence of UV/Vis-radiation.

### FIELD OF THE INVENTION

The present invention relates to a coating composition, a coating obtained using the composition and the use of a UV-absorber and a pigment for protecting a substrate against UV/Vis-radiation.

### BACKGROUND OF THE INVENTION

Polymers based on aromatic epoxides, aromatic polyesters and aromatic (poly-)isocyanates are prone to damage by UV radiation. Such polymers are for example used as electrochemical (cathodical) deposition coating (EDC) in automotive coating. Automotive coatings are exposed to UV radiation for a long time. The EDC layer is usually directly applied onto the substrate (normally metal) and additional coating layers, such as one or more (usually two) base coats providing the desired colour and a clear coat are applied subsequently.

A damage and subsequent degradation of the EDO-layer by UV radiation would cause the coating to chip off and, thus, needs to be avoided. Usually a filler layer is applied onto the EDC-layer for UV/Vis-protection. However, it is desired to avoid the filler layer for economic purposes. Nevertheless the UV/Vis-protection needs to be maintained. The UV protection, thus, needs to be provided by the other layers present, e.g. the base coat layers. The requirements of the car manufacturers as to the extent of the UV/Vis protection differ between the manufacturers. Some manufacturers require a maximum transmittance through the coating of 0.25% up to a wavelength range of 500 nm. Moreover the coating layers should be thin, usually 20 μm or less. Depending on the colour shade, e.g. blue, red, silver white and pastel shades, the desired UV-protection cannot be obtained by the corresponding colour pigment—the pigment providing the colour of the coating—as such or only using high (and, thus undesired) pigments loads and/or layer thicknesses. For example, blue pigmented coatings usually already start to exceed the desired transmission starting at 360 nm and white pigmented layers starting at 400 nm upwards.

Thus, high UV/Vis-protection without a filler layer and without requiring high pigment loads and/or layer thicknesses is desired.

It has been surprisingly found that excellent UV protection can be obtained at low film builds by using a UV absorber in combination with a pigment.

### SUMMARY OF THE INVENTION

The present invention therefore provides a coating composition comprising
one or more compound(s) (A) according to the following formula (I)

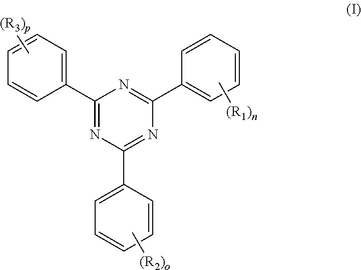

wherein
each R₁, R₂ and R₃ is independently selected from
   —OR₄, R₄ being independently hydrogen or a C₁ to C₂₄ hydrocarbyl group optionally containing heteroatoms; or
   C₁ to C₂₄ hydrocarbyl groups optionally containing heteroatoms;
n is 2, 3, 4 or 5;
o is 2, 3, 4 or 5;
p is 2, 3, 4 or 5;
with the proviso that at least one of R₁ and at least one of R₂ and at least one of R₃ is —OR₄
and
a pigment (B) characterized by a minimum integrated transmittance within the range of 380 to 600 nm.

It has been surprisingly found that with the above combination excellent UV absorption up to about 500 nm or above can be obtained while applying a low dry film thickness (20 μm or less). In addition, it has been surprisingly found that excellent UV/Vis-protection over the range of 280 to about 450 nm and even up to greater than 500 nm can be obtained by the combination of components (A) and (B) according to the invention. Furthermore, pigments can be used which do not need to cover the entire range of 280 to about 450 nm opening the possibility to use pigments hitherto not considered suitable for UV protection. Thus, the coating composition according to the present invention is particularly suitable as first base coat whereon a second base coat usually providing the desired colour shade is applied. Alternatively the composition of the present invention can be used as only base coat. Moreover, an undesired high pigmentation or layer thickness can be avoided. Particularly, the layer thicknesses applied can be harmonized allowing shorter production cycles. Hence, long drying times (which would be necessary in case of thicker layers) or even varying drying times dependent on the different layer thicknesses caused by the specific, desired colour are not required. Moreover the composition is stable towards UV/Vis radiation and allows low transmittance above 450 nm and even up to greater than 500 nm.

The minimum integrated transmittance usually takes absorption, reflection and scattering into account and, thus, allows a more reliable characterisation of the pigment. The exact determination of the minimum integrated transmittance is described in the experimental part.

The present invention is further directed to a coating obtained by applying the composition according to the invention on a substrate.

The present invention is furthermore directed to the use of components (A) and (B) for protecting a substrate against UV/Vis-radiation.

The present invention is furthermore directed to a process for the stabilization of a coating against the deleterious influence of UV/Vis-radiation, which comprises applying the coating composition according to the invention to a substrate.

### DETAILED DESCRIPTION OF THE INVENTION

The minimum integrated transmittance of the pigment (B) denotes the absolute minimum of the integrated transmittance curve.

UV/Vis-radiation denotes light within the wavelength range of 280 to 600 nm.

The coating composition is preferably an automotive coating composition.

As already outlined above, compound (A) is according to the following formula (I)

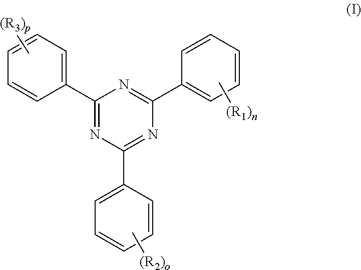

wherein
each R₁, R₂ and R₃ is independently selected from
   —OR₄, R₄ being independently hydrogen or a C₁ to C₂₄ hydrocarbyl group optionally containing heteroatoms; or
   C₁ to C₂₄ hydrocarbyl groups optionally containing heteroatoms;
n is 2, 3, 4 or 5, preferably 2, 3 or 4 and most preferably 2 or 3;
o is 2, 3, 4 or 5, preferably 2, 3 or 4 and most preferably 2 or 3;
p is 2, 3, 4 or 5, preferably 2, 3 or 4 and most preferably 2 or 3;
with the proviso that at least one of R₁ and at least one of R₂ and at least one of R₃ is —OR₄.

The coating composition according to the present invention can comprise one or more compounds (A). For example during synthesis of the compound (A) a mixture of isomers may be obtained. It is also possible to use different compounds (A) within the coating composition according to the present invention.

In the present invention “one or more compounds (A)” denotes that up to 7 different compounds (A) may be present in the coating composition according to the present invention, preferably up to 5.

In one embodiment only one compound (A) is present in the coating composition according to the present invention.

Preferably, compound (A) is free of metals.

In case R₄ is different from hydrogen, R₄ is usually bound to the oxygen atom via a carbon atom in —OR₄.

In case R₁, R₂ and/or R₃ are C₁ to C₂₄ hydrocarbyl groups containing heteroatoms or any preferred embodiment thereof the atom of R₁, R₂ and/or R₃ bound to the aromatic rings depicted in formula I) above are carbon atoms. Thus, for example R₁, R₂ and/or R₃ may be a —CH₂—O— CH₃ residue but not a —O—CH₂—CH₃ residue.

In case R₄ is a C₁ to C₂₄ hydrocarbyl group optionally containing heteroatoms, R₄ preferably does not contain more than 5 heteroatoms, more preferably not more than 3 heteroatoms, even more preferably not more than one heteroatom. In a preferred embodiment R₄ does not contain heteroatoms.

R₄ is preferably independently selected from hydrogen or a C₁ to C₂₀ hydrocarbyl group optionally containing heteroatoms, more preferably independently selected from hydrogen or a C₁ to C₁₅ hydrocarbyl group optionally containing heteroatoms, even more preferably independently selected from hydrogen or a C₁ to C₁₅ hydrocarbyl group free from heteroatoms, even more preferably independently selected from hydrogen or a C₁ to C₁₅ alkyl group free from heteroatoms. It is particularly preferred that R₄ is independently selected from hydrogen or a C₁ to C₁₂ hydrocarbyl group free from heteroatoms, more preferably independently selected from hydrogen or a C₁ to C₁₂ alkyl group free from heteroatoms and most preferably independently selected from hydrogen or a C₁ to C₆ alkyl group free from heteroatoms, e.g. methyl, ethyl, n-propyl, iso-propyl, n-butyl, sec-butyl, tert.-butyl. Methyl and hydrogen are particularly preferred.

In case R₁, R₂ and/or R₃ are C₁ to C₂₄ hydrocarbyl groups optionally containing heteroatoms, each of R₁, R₂ and/or R₃ preferably does not contain more than 5 heteroatoms, more preferably not more than 3 heteroatoms, even more preferably not more than one heteroatom. In a preferred embodiment each of R₁, R₂ and/or R₃ does not contain heteroatoms.

In case R₁, R₂ and/or R₃ are C₁ to C₂₄ hydrocarbyl groups optionally containing heteroatoms, preferably R₁, R₂ and/or R₃ are independently selected from C₁ to C₂₀ hydrocarbyl groups optionally containing heteroatoms, more preferably independently selected from C₁ to C₁₅ hydrocarbyls group optionally containing heteroatoms, even more preferably independently selected from C₁ to C₁₅ hydrocarbyl groups free from heteroatoms and most preferably independently selected from C₁ to C₁₅ alkyl groups free from heteroatoms. It is particularly preferred that R₁, R₂ and/or R₃ are independently selected from C₁ to C₁₂ hydrocarbyl groups free from heteroatoms, more preferably independently selected from C₁ to C₁₂ alkyl groups free from heteroatoms and most preferably independently selected from C₁ to C₆ alkyl groups free from heteroatoms, e.g. methyl, ethyl, n-propyl, iso-propyl, n-butyl, sec-butyl, tert.-butyl. Methyl is particularly preferred.

In case heteroatoms are present in R₁, R₂, R₃ and R₄ including preferred embodiments thereof, these heteroatoms are preferably independently selected from N, S, P and O, more preferably independently selected from N and O and most preferably are O.

Preferably, in component (A) according to formula (I), n=o=p, more preferably n=o=p and are selected from 2 or 3.

In case R₁, R₂, R₃ or R₄ are hydrocarbyl groups containing heteroatoms, the hydrocarbyl group containing heteroatoms may independently be selected from hydrocarbyl groups, e.g. aliphatic or aromatic groups, which
a) are substituted by one or more usually not more than five, preferably not more than three of —OH, —COO—R₁₀, —OCO—R₁₁, —OR₁₀, —NCO and/or —NH₂ groups
   with R₁₀ being independently selected from hydrocarbyl groups, preferably independently selected from alkyl or alkenyl groups, more preferably independently selected from alkyl groups, provided that the total number of carbon atoms present in R₁, R₂, R₃ and R₄ is within the range specified above, e.g. may be alkyl, alkenyl or aromatic;
   with R₁₁ being independently selected from hydrogen or hydrocarbyl groups, preferably independently selected from alkyl or alkenyl groups, more preferably independently selected from alkyl groups, provided that the total number of carbon atoms present in R₁, R₂, R₃ and R₄ is within the range specified above, e.g. may be alkyl, alkenyl or aromatic;
b) are interrupted by one or more usually not more than five, preferably not more than three of —O—, —NH— and/or —NR₁₀— groups;
   with R₁₀ being independently selected from hydrocarbyl groups, preferably independently selected from alkyl or alkenyl groups, provided that the total number of carbon atoms present in R₁, R₂, R₃ and R₄ is within the range specified above, e.g. may be alkyl, alkenyl or aromatic;
c) are —C(O)—O—R₁₀, —C(O)—NHR₁₀, —C(O)—N(R₁₀)₂;
   with R₁₀ being independently selected from hydrocarbyl groups, preferably independently selected from alkyl or alkenyl groups, provided that the total number of carbon atoms present in R₁, R₂, R₃ and R₄ is within the range specified above, e.g. may be alkyl, alkenyl or aromatic.

When any of R₁₀ and/or R₁₁ are alkyl, they can independently be straight, branched chain or cyclic alkyl, said alkyl comprises within the limits of carbon atoms given, for example, methyl, ethyl, isopropyl, n-butyl, isobutyl, sec-butyl, tert-butyl, tert-amyl, 2-ethylhexyl, tert-octyl, lauryl, tert-dodecyl, tridecyl, n-hexadecyl, n-octadecyl, eicosyl, cyclopentyl, cyclohexyl, cycloheptyl, cyclooctyl or cyclododecyl more preferably methyl, ethyl, isopropyl, n-butyl, isobutyl, sec-butyl, tert-butyl, tert-amyl, 2-ethylhexyl, tert-octyl.

When any of R₁₀ and/or R₁₁ are alkenyl, which may independently be straight or branched chain alkenyl, such groups are within the limits of carbon atoms given, for example, allyl, pentenyl, hexenyl, doceneyl or oleyl. In case of C₂-C₁₈ alkenyl, preference is given to C₃-C₁₆ alkenyl, especially C₃-C₁₂ alkenyl, for example C₂-C₆ alkenyl.

Preferably, in component (A) according to formula (I) at least two of R₁ and at least two of R₂ and at least two of R₃ are —OR₄, more preferably in component (A) according to formula (I) at least two of R₁ and at least two of R₂ and at least two of R₃ are —OR₄ and n, o and p are independently selected from 2 or 3.

Preferably, component (A) is a compound according to the following formula (II)

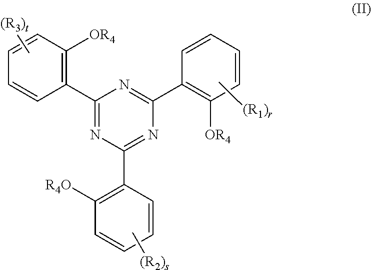

wherein
R₁, R₂, R₃ and R₄ are as defined above.
r is 1, 2, 3 or 4, preferably 1, 2 or 3 and most preferably 1 or 2;
s is 1, 2, 3 or 4, preferably 1, 2 or 3 and most preferably 1 or 2;
t is 1, 2, 3 or 4, preferably 1, 2 or 3 and most preferably 1 or 2;
preferably with the proviso that at least one of R₁ and at least one of R₂ and at least one of R₃
is —OR₄

Preferably, in component (A) according to formula (II), r=s=t, more preferably r=s=t and are selected from 1 or 2.

The preferred features for R₁, R₂, R₃ and R₄ according to formula (I) are also preferred features for R₁, R₂, R₃ and R₄ according to formula (II).

More preferably, component (A) is a compound according to the following formula (III)

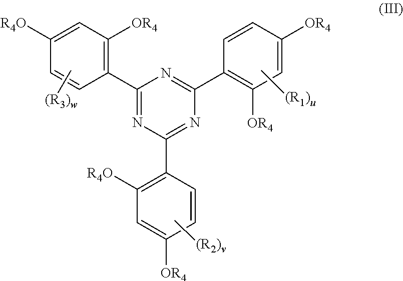

wherein
R₁, R₂, R₃ and R₄ are as defined above including all preferred features thereof.
u is 0, 1, 2 or 3, preferably 0, 1 or 2 and most preferably 0 or 1;
is 0, 1, 2 or 3, preferably 0, 1 or 2 and most preferably 0 or 1;
w is 0, 1, 2 or 3, preferably 0, 1 or 2 and most preferably 0 or 1;

In one variant u=v=w, more preferably u=v=w and are selected from 0 or 1.

In case R₁, R₂ and/or R₃ are present they are preferably present on the carbon atom adjacent to both carbon atoms which bear the OR₄-groups.

Particularly preferred are compounds according to formula (III) wherein
u is 0 or 1;
is 0 or 1;
w is 0 or 1;
preferably u=v=w;
in case R₁, R₂ and/or R₃ are present they are preferably present on the carbon atom adjacent to both carbon atoms which bear the OR₄-groups;
R₄ being free of heteroatoms and independently selected from hydrogen or C₁ to C₁₂ hydrocarbyl group, preferably independently selected from hydrogen or C₁ to C₁₂ alkyl groups, more preferably independently selected from hydrogen or C₁ to C₆ alkyl groups, e.g. methyl, ethyl, n-propyl, iso-propyl, n-butyl, sec-butyl, tert.-butyl whereby methyl and hydrogen are particularly preferred;
any of R₁, R₂, R₃, if present, being free of heteroatoms and independently selected from C₁ to C₁₅ hydrocarbyl groups, preferably independently selected from C₁ to C₁₅ alkyl groups, e.g. methyl, ethyl, n-propyl, iso-propyl, n-butyl, sec-butyl, tert.-butyl whereby methyl is particularly preferred.

Particularly suitable compounds are as follows.

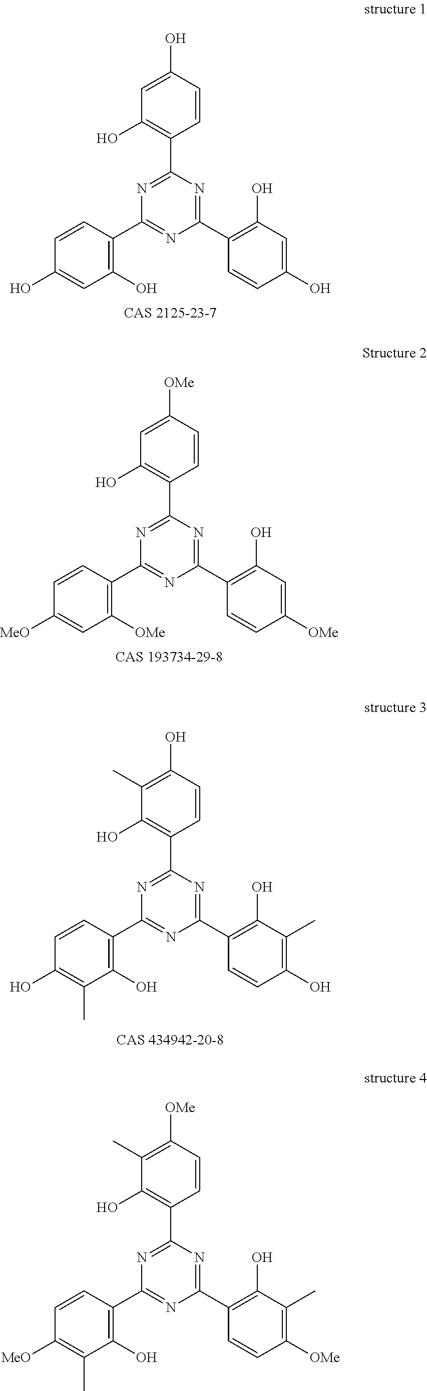 [SMILES: Oc1ccc(-c2nc(-c3ccc(O)cc3O)nc(-c3ccc(O)cc3O)n2)c(O)c1 ; COc1ccc(-c2nc(-c3ccc(OC)cc3O)nc(-c3ccc(OC)cc3OC)n2)c(O)c1 ; Cc1c(O)ccc(-c2nc(-c3ccc(O)c(C)c3O)nc(-c3ccc(O)c(C)c3O)n2)c1O ; COc1ccc(-c2nc(-c3ccc(OC)c(C)c3O)nc(-c3ccc(OC)c(C)c3O)n2)c(O)c1C]

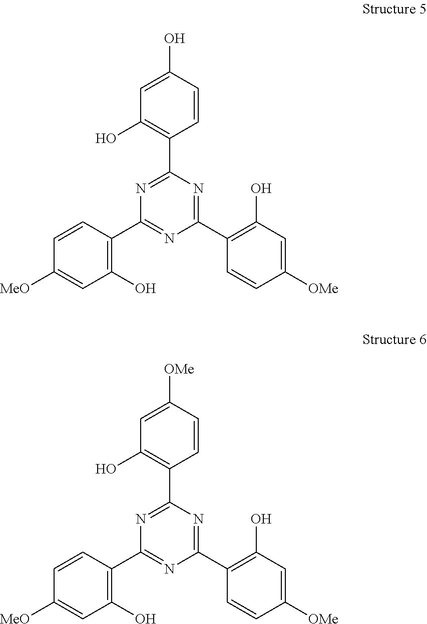 [SMILES: COc1ccc(-c2nc(-c3ccc(O)cc3O)nc(-c3ccc(OC)cc3O)n2)c(O)c1 ; COc1ccc(-c2nc(-c3ccc(OC)cc3O)nc(-c3ccc(OC)cc3O)n2)c(O)c1]

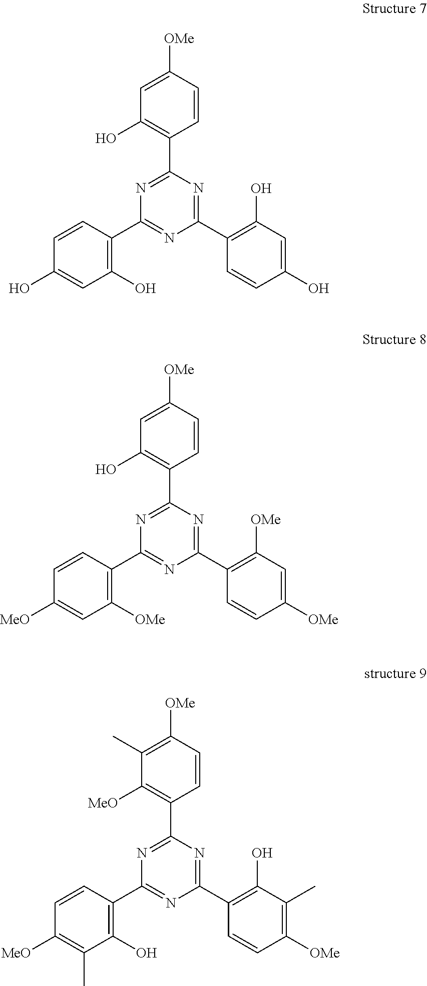 [SMILES: COc1ccc(-c2nc(-c3ccc(O)cc3O)nc(-c3ccc(O)cc3O)n2)c(O)c1 ; COc1ccc(-c2nc(-c3ccc(OC)cc3OC)nc(-c3ccc(OC)cc3OC)n2)c(O)c1 ; COc1ccc(-c2nc(-c3ccc(OC)c(C)c3O)nc(-c3ccc(OC)c(C)c3OC)n2)c(O)c1C]

The synthesis of structures 1 to 9 is described in the experimental part.

As outlined above, pigment (B) has a minimum of the integrated transmittance within the range of 380 to 600 nm, preferably within the range of 400 to 500 nm.

Preferably, the pigment (B) is selected from the color range from yellow to red, more preferably represented by the following classes or mixtures thereof:
isoindolines: C.I. PY 139 (e.g. Paliotol Yellow L 2140 HD, Paliotol Yellow L 2146 HD, Paliotol Yellow L 1970, Paliotol Yellow L 1820) or C.I. PY 185 e.g. Paliotol Yellow L 1155),
isoindolinones: C.I. PY 110 (e.g. Irgazin Yellow L 2040) or C.I. PY 109 (e.g. Irgazin Yellow L 1030)
benzimididazolones: C.I. PY 151 (e.g. Cromophtal Yellow L 1061 HD) or C.I. PY 154 (e.g. Cromophtal Yellow L 1084),
quinophthalones: C.I. PY 138 (e.g. Paliotol Yellow L 0962 HD, Paliotol Yellow L 0960 HD),
azomethine Cu-complexes: C.I. PY 129 (e.g. Irgazin Yellow L 0800),
bismuth vanadate: C.I. PY 184 (e.g. Sicopal Yellow L 1100, Sicopal Yellow L 1110, Sicopal Yellow L 1120, Sicopal Yellow L 1600),
mixed metal oxides: C.I. PY 53 (e.g. Ni-titanate such as Sicotan Yellow L 1010, Sicotan Yellow L 1012),
iron oxides; C.I. PY 42 (e.g. Sicotrans Yellow L 1916, Sicotrans Yellow L 1915) or C.I. PY 101 (e.g. Sicotrans Red L 2817) and
hybrid pigments (blends of organic and inorganic pigments) (e.g. Paliotan Yellow L 1145, Paliotan Yellow L 1645, Paliotan Yellow L 1945, Paliotan Yellow L 2045)

Particularly preferred thereof are isoindolines or quinophthalones.

In the composition according to the present invention components (A) and/or (B) are preferably each present in an amount of 0.1 to 30 wt. %, more preferably in an amount of 0.3 to 15 wt. % and most preferably in an amount of 0.5 to 5 wt. % based on the total solids content of the composition.

Another aspect of the instant invention is a coating, preferably an automotive coating, obtained by applying a coating composition, preferably an automotive coating composition according to the present invention on a substrate.

Such substrates are for example glass, metal, wood, plastic or ceramic materials, especially metal. Or such a substrate is another coating layer, preferably another automotive coating layer applied on such a substrate. Thus, the coating composition of the present invention is, for example, also suitable for repair coating.

Another coating layer, preferably another automotive coating layer, applied on a substrate as outlined above is preferred. Preferably, this another coating layer is a primer applied to a metal substrate.

Suitable primers are all commonly employed primers, particularly primers normally used for coating metallic substrates. Where the coating of the invention is used to coat other substrates, such as plastics, for example, the coating compositions customary for priming those substrates are used.

The primers used particularly for steel and similar metals are usually aqueous coating materials having a solids content of generally 10% to 25% by weight. They generally include at least one binder, at least one crosslinking agent, pigments if desired, and further customary auxiliaries and additives, if desired. Preference is given to using electrophoretically depositable coating materials, known as electrocoat materials, particularly cathodically depositable electrocoat materials, as primer. Suitability is also possessed, however, by, for example, primers which can be applied by means of the technique known as coil coating. For substrates of aluminum the primers (G) used generally comprise aluminum oxide layers produced by anodic oxidation.

The electrocoat materials usually comprise binders which carry ionic substituents or substituents which can be converted into ionic groups, and also carry groups capable of chemical crosslinking. The ionic groups may be anionic groups or groups which can be converted into anionic groups, COOH groups for example, or cationic groups or groups which can be converted into cationic groups, examples being amino, ammonium, quaternary ammonium, phosphonium and/or sulfonium groups. Preference is given to using binders containing basic groups, especially nitrogen-containing basic groups. These groups may be in quaternized form or are converted into ionic groups with customary neutralizing agents, examples being organic monocarboxylic acids, such as formic, acetic or lactic acid, for example.

Suitable anodically depositable electrocoat materials are known and are described for example in DE-A-28 24 418. They usually include self-crosslinking or externally crosslinking binders based on polyesters, epoxy resins, poly(meth)acrylates, maleate oils or polybutadiene oils which carry anionic groups, such as —COOH, —SO₃H and/or —PO₃H₂ groups, and also customary crosslinkers, such as triazine resins, blocked polyiso-cyanates or crosslinkers which carry transesterifiable groups, for example.

Suitable cathodically depositable electrocoat materials are likewise known and are described for example in EP-B 0 241 476, WO 91/09917, EP-B-0 920 480, EP-B 0 961 797, WO 2003/068418 and WO 2004/018580. They usually include self-crosslinking or externally crosslinking binders based on polyesters, epoxy resins, epoxy resins having terminal double bonds or OH groups, poly(meth)acrylates, polyurethane resins or polybutadiene resins which carry cationic groups, such as primary, secondary or tertiary amino groups, which have been neutralized with an organic acid, and also include customary crosslinkers, such as triazine resins, blocked polyisocyanates, amino resins, polyepoxide compounds or crosslinkers which carry transesterifiable groups or double bonds, for example.

Cathodically depositable electrocoat materials applied to a metal substrate are preferred.

Particular preference is given to using the cathodically depositable electrocoat materials, e.g. as described in EP-B-0 961 797, which comprise an aqueous binder dispersion based on epoxy resins which contain ammonium groups and are obtainable by
(I) reacting one or more diepoxy resins (a) with one or more mono- and/or diphenols (b) to give an intermediate (I),
(II) reacting the intermediate (I) with one or more amines to give an epoxide-amine adduct (A),
(III) subsequently or simultaneously reacting the secondary hydroxyl groups formed during the reaction of (a) and (b) with the epoxide groups of the epoxide-amine adduct (A),
(IV) adding at least one crosslinking agent,
(V) neutralizing, and
(VI) dispersing the resulting mixture in water.

Preferably the coating, obtained by applying the coating composition according to the invention on a substrate, is applied at a dry film thickness of 30 μm or less, preferably 20 μm or less.

Most preferably, the coating is an automotive coating and comprises the following layers
(a) a primer, preferably an elecrophoretically depositable material, particularly a cathodically deposited coating as defined in the present invention, applied to a metal substrate;
(b) at least one subsequent coating layer comprising, preferably consisting of, the automotive coating composition according to the present invention adhering to layer (a);
(c) optionally one or more, preferably one, further coating layer usually being free of components (A) and/or (B), preferably being free of components (A) and (B);
(d) a clear top coating containing one or more UV-absorbers different from those of formula (I) and optionally further light stabilizers.

In case (c) is not present, layer (b) is preferably directly next to layer (a) and layer (d) is directly next to layer (b). In case (c) is present, layer (b) is preferably directly next to layer (a), layer (c) is directly next to layer (b) and layer (d) is directly next to layer (c).

Preferred features of the composition according to the present invention are also preferred features of the automotive coating according to the invention and vice versa.

The coating layer (b) is preferably applied at a dry film thickness of 30 μm or less, preferably 20 μm or less. Usually the dry film thickness of coating layer (b) is at least 3 μm. The coating layer (a) is preferably applied at a dry film thickness of 35 μm or less, preferably 30 μm or less. Usually the dry film thickness of coating layer (a) is at least 10 μm.

The coating layer (d) is preferably applied at a dry film thickness of 50 μm or less, preferably 40 μm or less. Usually the dry film thickness of coating layer (d) is at least 30 μm.

Layer (c), if present, is a usual automotive coating layer as known in the art. Layer (c) if present, usually contains pigments. The coating layer (c), if present, is preferably applied at a dry film thickness of 30 μm or less, preferably 20 μm or less. Usually the dry film thickness of coating layer (c), if present, is at least 3 μm.

For example, in such an automotive coating, the metal substrate is pretreated in e.g. a customary zinc phosphate bath.

For instance, the automotive coating according to the present invention, is applied over a substrate, which is sensitive to electromagnetic radiation both in the UV range (280 to 380 nm) and furthermore at wavelengths greater than 380 nm.

A typical sensitive substrate is, for example, a cathodically deposited coating applied to a metal substrate. Such coatings are typically used in the automotive industry.

Under sensitive to electromagnetic radiation of wavelengths greater than 380 nm there is understood UV or visible light, for example, in the wavelength range up to 600 nm, preferably up to 500 nm and in particular up to 450 nm.

The coating composition, especially the automotive coating composition, according to the present invention usually comprises a polymeric resin or the precursors thereof, normally denoted binder.

Resins used in coatings, preferably automotive coatings, are typically crosslinked polymers, for example, derived from aldehydes on the one hand and phenols, ureas and melamines on the other hand, such as phenol/formaldehyde resins, urea/formaldehyde resins and melamine/formaldehyde resins.

Also useful are unsaturated polyester resins derived from copolyesters of saturated and unsaturated dicarboxylic acids with polyhydric alcohols and vinyl compounds as crosslinking agents, and also halogen-containing modifications thereof of low flammability. Preferably used are crosslinkable acrylic resins derived from substituted acrylates, for example epoxy acrylates, urethane acrylates or polyester acrylates.

Also possible are alkyd resins, polyester resins and acrylate resins crosslinked with melamine resins, urea resins, isocyanates, isocyanurates, polyisocyanates or epoxy resins. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.

The coating material may also be a radiation curable composition containing ethylenically unsaturated monomers or oligomers and a polyunsaturated aliphatic oligomer.

The alkyd resin lacquers which can be stabilized against the action of light in accordance with the instant invention are the conventional stoving lacquers which are used in particular for coating automobiles (automobile finishing lacquers), for example lacquers based on alkyd/melamine resins and alkyd/acrylic/melamine resins (see H. Wagner and H. F. Sarx, “Lackkunstharze” (1977), pages 99-123). Other crosslinking agents include glycouril resins, blocked isocyanates or epoxy resins.

It is also to be noted that the compounds (A) and (B) according to the present invention are applicable for use in non-acid catalyzed thermoset resins such as epoxy, epoxy-polyester, vinyl, alkyd, acrylic and polyester resins, optionally modified with silicon, isocyanates or isocyanurates. The epoxy and epoxy-polyester resins are crosslinked with conventional crosslinkers such as acids, acid anhydrides, amines and the like. Correspondingly, the epoxide may be utilized as the crosslinking agent for various acrylic or polyester resin systems that have been modified by the presence of reactive groups on the backbone structure.

When water-soluble, water miscible or water dispersible coatings are desired, ammonium salts of acid groups present in the resin are formed. Powder coating composition can be prepared by reacting glycidylmethacrylate with selected alcohol components.

Suitable binders, crosslinking agent and customary auxiliaries and additives inter alia for layers (b), (c) and (d) are described in US 2009/317629 and WO 2006/131469 which are herewith incorporated by reference in their entirety.

The coating composition according to the present invention may comprise further additives, e.g. antioxidants, UV absorbers and light stabilizers different from the compounds according to the present invention, metal deactivators, nucleating agents and/or fillers and reinforcing agents.

Suitable UV absorbers can be selected from the class of hydroxy-phenyl-benzotriazioles, hydroxy-phenyl-triazines, hydroxyl-benzophenones, oxanilides, cyanoacrylates or malonates, sterically hindered amines compounds and combinations thereof.

Suitable hydroxy-phenyl-benzotriazioles are, for example, 2-(2′-hydroxy-5′-methylphenyl)-benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis-(alpha,alpha-dimethylbenzyl)-2′-hydroxyphenyl)benzotriazole, 2-(3′-tert-butyl-2′-hydroxy-5′-(2-octyloxycarbonylethyl)phenyl)-5-chloro-benzotriazole, 2-(3′-tert-butyl-5′-[2-(2-ethylhexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylene-bis[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH₂CH₂—COO—CH₂CH₂₂, where R=3′-tert-butyl-4′-hydroxy-5′-2H-benzotriazol-2-ylphenyl, 2-[2′-hydroxy-3′-(alpha, alpha-dimethylbenzyl)-5′-(1,1,3,3-tetramethylbutyl)-phenyl]-benzotriazole; 2-[2′-hydroxy-3′-(1,1,3,3-tetramethylbutyl)-5′-(alpha, alpha-dimethylbenzyl)-phenyl]benzotriazole.

Suitable hydroxy-phenyl-triazine are, for example, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyl-oxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethyl-phenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2-(2-hydroxyphenyl)-4-(4-methoxyphenyl)-6-phenyl-1,3,5-tri-azine, 2-{2-hydroxy-4-[3-(2-ethylhexyl-1-oxy)-2-hydroxypropyloxy]phenyl}-4,6-bis(2,4-di-methylphenyl)-1,3,5-triazine.

Suitable hydroxyl-benzophenones are, for example, 4-hydroxy, 4-methoxy, 4-octyloxy, 4-decyl-oxy, 4-dodecyloxy, 4-benzyloxy, 4,2′,4′-trihydroxy and 2′-hydroxy-4,4′-dimethoxy derivatives.

Suitable oxanilides are, for example, 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

Suitable cyanoacrylates are, for example, ethyl alpha-cyano-beta,beta-diphenylacrylate, isooctyl alpha-cyano-beta,beta-diphenylacrylate, methyl alpha-carbomethoxycinnamate, methyl alpha-cyano-alpha-methyl-p-methoxycinnamate, butyl alpha-cyano-alpha-methyl-p-methoxy-cinnamate, methyl alpha-carbomethoxy-p-methoxycinnamate, N-(beta-carbomethoxy-beta-cyanovinyl)-2-methylindoline, neopentyl tetra(alpha-cyano-beta, beta-di-phenylacrylate.

Suitable malonates are, for example 4-methoxy-benzylidene-di-(1-methylbutyl)malonate, 4-methoxy-benzylidene-di-isopropyl-malonate, 4-ethoxy-benzylidene-di-di-isopropyl-malonate, 4-n-propoxybenzylidene-di-isopropyl-malonate, 4-n-butoxybenzylidene-di-isopropyl-malonate, 4-methoxybenzylidene-di-tert-butyl-malonate, 4-methoxybenzylidene-di-(1,1-dimethylpropyl)-malonate.

Suitable sterically hindered amine compounds are, for example, bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-di-chloro-1,3,5-triazine, tris(2,2,6,6-tetramethyl-4-piperidyl)nitrilotriacetate, tetrakis(2,2,6,6-tetra-methyl-4-piperidyl)-1,2,3,4-butanetetracarboxylate, 1,1′-(1,2-ethanediyl)-bis(3,3,5,5-tetrame-thylpiperazinone), 4-benzoyl-2,2,6,6-tetramethylpiperidine, 4-stearyloxy-2,2,6,6-tetramethyl-piperidine, bis(1,2,2,6,6-pentamethylpiperidyl)-2-n-butyl-2-(2-hydroxy-3,5-di-tert-butylbenzyl)-malonate, 3-n-octyl-7,7,9,9-tetramethyl-1,3,8-triazaspiro[4.5]decane-2,4-dione, bis(1-octyl-oxy-2,2,6,6-tetramethylpiperidyl)sebacate, bis(1-octyloxy-2,2,6,6-tetramethylpiperidyl)succinate, linear or cyclic condensates of N,N′-bis(2,2,6,6-tetramethyl-4-piperidyl)hexamethylene-diamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)-ethane, the condensate of 2-chloro-4,6-di-(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetrame-thyl-1,3,8-triazaspiro[4.5]decane-2,4-dione, 3-dodecyl-1-(2,2,6,6-tetramethyl-4-piperidyl)pyr-rolidine-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensate of N,N′-bis(2,2,6,6-tetramethyl-4-piperidyl)hexamethylenediamine and 4-cyclohexylamino-2,6-dichloro-1,3,5-triazine, a condensate of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); a condensate of 1,6-hexanediamine and 2,4,6-trichloro-1,3,5-triazine as well as N,N-dibutylamine and 4-butylamino-2,2,6,6-tetramethylpiperidine (CAS Reg. No. [192268-64-7]); N-(2,2,6,6-tetramethyl-4-piperidyl)-n-dodecylsuccinimide, N-(1,2,2,6,6-pentamethyl-4-piperidyl)-n-dodecylsuccinimide, 2-undecyl-7,7,9,9-tetramethyl-1-oxa-3,8-diaza-4-oxo-spiro-[4,5]decane, a reaction product of 7,7,9,9-tetramethyl-2-cycloundecyl-1-oxa-3,8-diaza-4-oxo-spiro-[4,5]decane and epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexa-methylenediamine, a diester of 4-methoxymethylenemalonic acid with 1,2,2,6,6-pentamethyl-4-hydroxypiperidine, poly[methylpropyl-3-oxy-4-(2,2,6,6-tetramethyl-4-piperidyl)]siloxane, a reaction product of maleic acid anhydride-alpha-olefin copolymer with 2,2,6,6-tetramethyl-4-ami-nopiperidine or 1,2,2,6,6-pentamethyl-4-aminopiperidine, 2,4-bis[N-(1-cyclohexyloxy-2,2,6,6-tetramethylpiperidine-4-yl)-N-butylamino]-6-(2-hydroxyethyl)amino-1,3,5-triazine, 1-(2-hydroxy-2-methylpropoxy)-4-octadecanoyloxy-2,2,6,6-tetramethylpiperidine, 5-(2-ethylhexanoyl)-oxymethyl-3,3,5-trimethyl-2-morpholinone, SANDUVOR® (Clariant; CAS Reg. No. 106917-31-1], 5-(2-ethylhexanoyl)oxymethyl-3,3,5-trimethyl-2-morpholinone, the reaction product of 2,4-bis-[(1-cyclohexyloxy-2,2,6,6-piperidine-4-yl)butylamino]-6-chloro-s-triazine with N,N′-bis(3-aminopropyl)ethylenediamine), 1,3,5-tris(N-cyclohexyl-N-(2,2,6,6-tetramethylpiperazine-3-one-4-yl)amino)-s-triazine, 1,3,5-tris(N-cyclohexyl-N-(1,2,2,6,6-pentamethylpiperazine-3-one-4-yl)-amino)-s-triazine.

The coating composition according to the present invention may additionally comprise one or more of the above UV absorbers selected from the class of hydroxy-phenyl-benzotriaziole or hydroxy-phenyl-triazine or hydroxyl-benzophenone or oxanilide or cyanoacrylate or malonate and combinations thereof.

The coating composition according to the present invention may additionally comprise a sterically hindered amine compound as defined above.

In one embodiment, the coating composition according to the present invention is additionally comprising a sterically hindered amine compound as defined above and is additionally comprising one or more of the above UV absorbers selected from the class of hydroxy-phenyl-benzotriazioles, hydroxy-phenyl-triazines, hydroxyl-benzophenones, oxanilides, cyanoacrylates, malonates and combinations thereof.

In another embodiment the coating composition according to the present invention does not comprise UV absorbers different from compound (A) according to the present invention.

The present invention is furthermore directed to the use of
one or more compound(s) (A) according to the following formula (I)

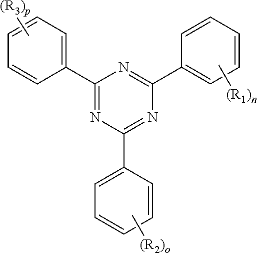

wherein
each R₁, R₂ and R₃ is independently selected from
   —OR₄, R₄ being hydrogen or a C₁ to C₂₄ hydrocarbyl group optionally containing heteroatoms; or
   C₁ to C₂₄ hydrocarbyl groups optionally containing heteroatoms;
n is 2, 3, 4 or 5;
o is 2, 3, 4 or 5;
p is 2, 3, 4 or 5;
with the proviso that at least one of R₁ and at least one of R₂ and at least one of R₃ is —OR₄
and
a pigment (B) which minimum integrated transmittance is within the range of 380 to 600 nm for protecting a substrate against UV/Vis-radiation.

As already outlined above, UV/Vis-radiation denotes light within the wavelength range of 280 to 600 nm.

Such a substrate is for example glass, metal, wood, plastic or ceramic materials, especially metal. Or such a substrate is another coating layer, preferably another automotive coating layer applied on such a substrate.

Another coating layer, preferably another automotive coating layer, applied on a substrate as outlined above is preferred. Preferably, this another coating layer is a primer applied to a metal substrate as defined in the present invention.

Preferred features of the coating composition according to the present invention including preferred features of components (A) and (B) are also preferred features of the use according to the present invention and vice versa.

A further aspect of the instant invention is a process for the stabilization of a substrate, against the deleterious influence of UV/Vis-radiation, which comprises applying the coating composition of the invention to the substrate.

Such a substrate is for example glass, metal, wood, plastic or ceramic materials, especially metal. Or such a substrate is another coating layer, preferably another automotive coating layer applied on such a substrate.

Another coating layer, preferably another automotive coating layer, applied on a substrate as outlined above is preferred. Preferably, this another coating layer is a primer applied to a metal substrate as defined in the present invention.

Most preference is given to a process wherein process comprises
(a) applying a primer, preferably an elecrophoretically depositable material, particularly a cathodically deposited coating, applied to a metal substrate
(b) applying the coating composition of the invention adhering to the layer (a) as defined in the present invention;
(c) optionally applying one or more, preferably one, further coating layer being free of components (A) and/or (B), preferably being free of components (A) and (B);
   and
(d) applying a clear top coating over the coating (b) adhering to coating (a) or to the coating (c), if present, adhering to the coating (b), which clear top coating contains one or more UV-absorbers different from those of formula (I) and optionally further light stabilizers.

By applying the coating composition of the invention adhering to the layer (a) as defined in the present invention in step b) the substrate is usually stabilized against the deleterious influence of UV/Vis-radiation,

In case (c) is not present, layer (b) is preferably directly next to layer (a) and layer (d) is directly next to layer (b). In case (c) is present, layer (b) is preferably directly next to layer (a), layer (c) is directly next to layer (b) and layer (d) is directly next to layer (c).

Clear coating layers are usually free of pigments.

Preferred features of the coating composition and use according to the present invention, including preferred features of components (A) and (B) are also preferred features of the process according to the present invention and vice versa.

In the following clauses preferred embodiments of the invention are described.

1. A coating composition comprising
one or more compound(s) (A) according to the following formula (I)

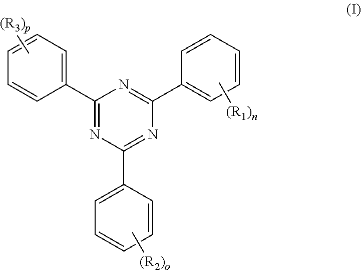

wherein
each R₁, R₂ and R₃ is independently selected from
   —OR₄, R₄ being independently hydrogen or a C₁ to C₂₄ hydrocarbyl group optionally containing heteroatoms; or
   C₁ to C₂₄ hydrocarbyl groups optionally containing heteroatoms;
n is 2, 3, 4 or 5;
o is 2, 3, 4 or 5;
p is 2, 3, 4 or 5;
with the proviso that at least one of R₁ and at least one of R₂ and at least one of R₃ is —OR₄
and
a pigment (B) which minimum integrated transmittance is within the range of 380 to 600 nm.
   2. The composition according to clause 1, wherein in component (A) at least two of R₁ and at least two of R₂ and at least two of R₃ are —OR₄.
   3. The composition according to clause 1 or 2, wherein component (A) is a compound according to the following formula (II)

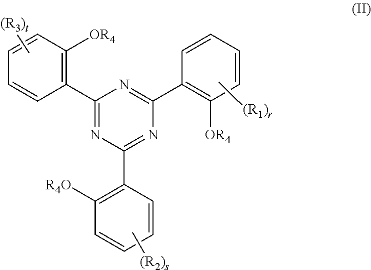

wherein
R₄ being hydrogen or a C₁ to C₂₄ hydrocarbyl group optionally containing heteroatoms;
each R₁, R₂ and R₃ is independently selected from
   —OR₄, R₄ being hydrogen or a C₁ to C₂₄ hydrocarbyl group optionally containing heteroatoms; or
   C₁ to C₂₄ hydrocarbyl groups optionally containing heteroatoms;
r is 1, 2, 3 or 4;
s is 1, 2, 3 or 4;
t is 1, 2, 3 or 4.
   4. The composition according to any one of the preceding clauses 1 to 3, wherein the pigment (B) is selected from the class of isoindolines, isoindolinones, benzimidazolones, quinophthalones, azomethine Cu-complexes, bismuth vanadate, mixed metal oxides or iron oxides or mixtures thereof.
   5. The composition according to clause 4, wherein the pigment (B) is selected from the class of isoindolines or quinophthalones.
   6. The composition according to any one of the preceding clauses 1 to 5, wherein components (A) and/or (B) are each present in an amount of 0.1 to 30 wt. %, preferably in an amount of 0.3 to 15 wt. % and most preferably in an amount of 0.5 to 5 wt. % based on the total solids content of the composition.
   7. The composition according to any one of the preceding clauses 1 to 6, additionally comprising one or more UV absorbers selected from the class of the hydroxy-phenyl-benzotriazioles, hydroxy-phenyl-triazines, hydroxyl-benzophenones, oxanilides, cyanoacrylates, malonates and combinations thereof.
   8. The composition according to any one of the preceding clauses 1 to 7, additionally comprising a sterically hindered amine compound.
   9. A coating obtained by applying the composition according to any one of the preceding clauses 1 to 8 on a substrate.
   10. The coating according to clause 9, whereby the coating is applied at a dry film thickness of 30 μm or less, preferably 20 μm or less.
   11. The coating according to clauses 9 to 10 wherein the substrate is glass, metal, wood, plastic or ceramic materials or another coating layer applied on such a substrate, preferably, the another coating layer is a primer and the substrate the primer is applied on is metal.

### 12. Use of

one more compound(s) (A) according to the following formula (I)

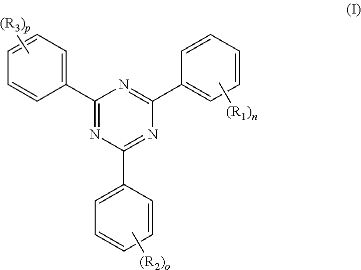

wherein
each R₁, R₂ and R₃ is independently selected from
   —OR₄, R₄ being hydrogen or a C₁ to C₂₄ hydrocarbyl group optionally containing heteroatoms; or
   C₁ to C₂₄ hydrocarbyl groups optionally containing heteroatoms;
n is 2, 3, 4 or 5;
o is 2, 3, 4 or 5;
p is 2, 3, 4 or 5;
with the proviso that at least one of R₁ and at least one of R₂ and at least one of R₃ is —OR₄
and
a pigment (B) which minimum integrated transmittance is within the range of 380 to 600 nm for protecting a substrate against UV/Vis-radiation.
   13. The use according to clause 12 wherein the substrate is glass, metal, wood, plastic or ceramic materials or another coating layer applied on such a substrate, preferably, the another coating layer is a primer and the substrate the primer is applied on is metal.
   14. A process for the stabilization of a substrate against the deleterious influence of UV/Vis-radiation, which comprises applying the coating composition according to clauses 1 to 8 to the substrate.
   15. The process of clause 14 wherein the coating is an automotive coating.
   16. The process according to clauses 14 to 15 wherein the substrate is glass, metal, wood, plastic or ceramic materials or another coating layer applied on such a substrate, preferably, the another coating layer is a primer and the substrate the primer is applied on is metal.

### EXPERIMENTAL PART

### Transmittance Determination

The UV/Vis transmittance spectra were recorded with a Perkin Elmer Lambda 650 S with 150 mm integrating sphere (Ulbricht sphere) using UV WinLab software. The Ulbricht sphere collects radiation scattered to different directions rather than directly transmitted radiation only. The application and use of integrating spheres is known in the art and can be looked up in literature like brochure “Application and use of integrating spheres with the Lambda 650 and 850 UV/Vis and Lambda 950 UV/Vis/NIR spectrophotometers” from Perkin Elmer. The sample preparation is described below.

### Sample Preparation for the Determination of the UV/VIS Transmittance Spectra of the Pigments (in a Clear Coat):

The pigment is added to formulation 1 described below (3 wt. % of the pigment on the solid content). The distribution of the pigment (dispersing) was conducted by adding the same weight amount of 2 mm glass beads and shaking of the mixture for 2 hours using a Scandex shaker (manufacturer: Lau GmbH). Subsequently the glass beads were filtered off. The formulation was applied (wire coater 50 μm) onto a glass plate resulting after cure (80°, 20 min) in a dry film thickness of 20 μm.

### Used Components:

### UV Absorber:

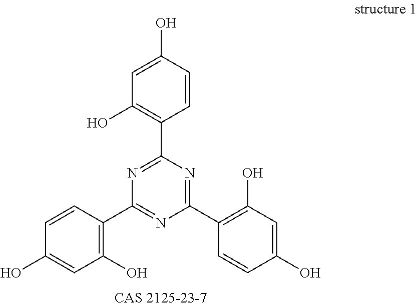 [SMILES: Oc1ccc(-c2nc(-c3ccc(O)cc3O)nc(-c3ccc(O)cc3O)n2)c(O)c1] [0214] Pigment 1: Pigment Yellow 138 (Quinophthalone); supplied by BASF SE
Pigment 2: Pigment Yellow 139 (Isoindoline); supplied by BASF SE
Tinuvin 123: Bis-(1-octyloxy-2,2,6,6-tetramethyl-4-piperidinyl) sebacate, CAS-no. 129757-67-1 supplied by BASF SE

The synthesis of structure 1 is described in EP 165 608.

### Example 1: Preparation of Structure 2

85 g potassium carbonate are added to 50 g of structure 1 in 380 ml DMF. While stirring the mixture is heated up to 80° C. and then 75 g dimethylsulfate are added dropwise. When the addition is finished stirring at 80° C. is continued for 3-4 hours, then heating is stopped. When the reaction mixture has cooled down to room temperature it is poured into 1 liter of ice cold water. The precipitate is filtered off, washed with water until the pH of the filtrate is neutral and dried in vacuo at 70° C. The crude product is recrystallized from 800 ml of hot dioxane to yield after filtration and drying in vacuo at 70° C. 40.5 g of structure 2 containing small amounts of structure 6 and structure 8. The melting range of the product is 210-220° C.

### Example 2: Preparation of Structure 3

25 g cyanurchloride (CAS 108-77-0) and 27.4 g aluminum trichloride are suspended in 475 ml of chlorobenzene. Then 58.9 g of 2-methyl-1,3-resorcinol (CAS 608-25-3) are added slowly while cooling with ice water. When the addition is completed the reaction mixture is heated within 30 minutes to 70° C. After stirring at 79° C. for additional 30 minutes the mixture is let cool to room temperature and then poured into a mixture of 150 ml of 2 N hydrochloric acid and 350 ml methanol containing some ice cubes. The precipitate is filtered off and dried in vacuo at 80° C. The crude product is recrystallized from DMF, washed with methanol and dryed in vacuo at 80° C. to yield 36.0 g of structure 3. The melting is above 395° C.

### Example 3: Preparation of Structure 4

31 g potassium carbonate are added to 20 g of structure 3 in 200 ml DMF. While stirring the mixture is heated up to 80° C. and then 25.4 g dimethylsulfate are added dropwise. When the addition is finished stirring at 80° C. is continued for 7 hours, then heating is stopped. When the reaction mixture has cooled down to room temperature it is poured into 1 liter of ice cold water. The precipitate is filtered off, washed with water until the pH of the filtrate is neutral and dried in vacuo at 70° C. Suspending the crude product first in 800 ml of hot dioxane and then in 300 ml of hot DMF yields after filtration and drying in vacuo at 80° C. 9.6 g of structure 4 containing small amounts of isomers of structure 4 and of structure 9. The product melts under decomposition at around 360° C.

### Example 4: Preparation of Mixture of Structures 5 and 6

35.8 g potassium carbonate are added to 50.0 g of structure 1 in 300 ml DMF. While stirring the mixture is heated up to 130° C. and stirred overnight. Then 31.1 g dimethylsulfate are added dropwise. When the addition is finished stirring at 130° C. is continued for 3 hours, then heating is stopped. When the reaction mixture has cooled down to room temperature it is poured into 1.5 liter of ice cold water. The precipitate is filtered off and the still wet filter cake is suspended in 150 ml of hot DMF. After stirring the suspension for 90 minutes at 130° C. and then cooling to room temperature the precipitate is filtered off and dried in vacuo at 80° C. to yield 15.9 g of a mixture, which consists mainly of ⅓ of structure 5 and ⅔ of structure 6, but is containing also isomers of structure 6 and a small amount unconverted structure 1.

### Example 5: Preparation of Mixture of Structures 1, 5 and 7

17.9 g potassium carbonate are added to 50.0 g of structure 1 in 300 ml DMF. While stirring the mixture is heated up to 70° C. and stirred for 4 h. Then 15.6 g dimethylsulfate are added dropwise. When the addition is finished stirring at 70° C. is continued for 3 hours, then heating is stopped. When the reaction mixture has cooled down to room temperature it is poured into 2.0 liter of ice cold water. The precipitate is filtered off and the still wet filter cake is suspended in hot water, is filtered off again, is washed with water until the pH of the filtrate is neutral and dried in vacuo at 80° C. to yield 44.8 g of a mixture, which consists mainly of about ⅓ of structure 1 and ½ of structure 7, ⅕ of structure 5 and a small amount of structure 6.

### Example 6: Preparation of Mixture of Structures 5, 6 and 7

11.1 g potassium carbonate are added to 32.0 g of the product of example 4 in 250 ml DMF. While stirring the mixture is heated up to 70° C. and stirred for 3 h. Then 9.6 g dimethylsulfate are added dropwise. When the addition is finished stirring at 70° C. is continued for 2 hours, then heating is stopped. When the reaction mixture has cooled down to room temperature it is poured into 1.5 liter of ice cold water. The precipitate is filtered off and dried in vacuo at 80° C. to yield 27.4 g of a product mixture. 15.0 g of this mixture are stirred with 150 ml of DMF at 130° C. for 16 h. After cooling to room temperature the mixture is poured into 500 ml of ice cold water. The precipitate is filtered off, washed with water until the pH of the filtrate is neutral and dried in vacuo at 80° C. to yield 11.3 g of a mixture, which consists of about ⅓ of each structure 5 and isomers, structure 6 and isomers and structure 7.

### Example 7: Preparation of Mixture of Structures 1, 2, 5, 6 and 7

46.0 g potassium carbonate are added to 50 g of structure 1 in 300 ml DMF. The mixture is heated up to 80° C. and stirred for 3 h. Then 38.9 g dimethylsulfate are added dropwise. When the addition is finished stirring at 80° C. is continued for 6 hours, then heating is stopped. When the reaction mixture has cooled down to room temperature it is poured into 1.5 liter of ice cold water. The precipitate is filtered off, and dried in vacuo. Suspending the crude product in 200 ml of hot dioxane yields after filtration and drying in vacuo at 80° C. 40.2 g of a mixture of about ¼ of structure, ⅖ of structure 5, ¼ of structure 5 and isomers thereof, plus little of an isomer of structure 5, little of structure 2 and little of unreacted starting material (structure 1). The product starts melting and decomposing at around 240° C.

### Example 8: Preparation of Mixture of Structures 2, 5, 6 and 7

59.7 g potassium carbonate are added to 50 g of structure 1 in 300 ml DMF. The mixture is heated up to 80° C. and stirred for 8 h. Then 49.8 g dimethylsulfate are added dropwise. When the addition is finished stirring at 80° C. is continued for 7 hours, then heating is stopped. When the reaction mixture has cooled down to room temperature it is poured into 1.5 liter of ice cold water. The precipitate is filtered off and washed with water until the pH of the filtrate is neutral. The precipitate is filtered off, and dried in vacuo at 70° C. to yield 49.1 g of crude product. The crude product is recrystallized from 100 ml of dioxane yielding a first product fraction of 13.2 g after drying in vacuo at 70° C. Concentrating the mother liquor of the recrystallization yields a second product fraction of 9.9 g after drying in vacuo at 70° C. The 2^{nd} fraction is a mixture of structure 6 and isomers thereof (˜⅖) structure 2 (˜⅖), structure 5 and isomers of thereof (˜⅙), plus little of structure 7, which starts melting at around 205° C. and decomposing at about 260° C.

The 1^{st} product fraction is suspended in 100 ml of hot dioxane and after cooling to room temperature filtered off and dried in vacuo at 70° C. to yield 9.1 g of a mixture of structure 2 (˜½) structure 6 and isomers thereof (˜⅓), structure 5 and isomers of thereof (˜⅙), plus little of structure 7, which starts melting at and decomposing at about 210° C.

### Example 9: Preparation of Mixture of Structures 2, 5, 6, 7 and 8

52.8 g potassium carbonate are added to 50 g of structure 1 in 300 ml DMF. The mixture is heated up to 135° C. and stirred for 16 h. Then 46.7 g dimethylsulfate are added dropwise. When the addition is finished stirring at 135° C. is continued for 3 hours, then heating is stopped. When the reaction mixture has cooled down to room temperature it is poured into 1.0 liter of ice cold water. The precipitate is filtered off and dried in vacuo at 70° C.t. The crude product is first suspended in 300 ml of hot dioxane, after cooling to room temperature filtered off and then suspended in 100 ml of hot DMF. After cooling to room temperature, filtration and drying in vacuo at 80° C. 40.2 g of product are obtained. The product is a mixture of about ⅔ of structure 2 and isomers thereof, ⅙ structure 6 and isomers thereof, ⅙ structure 5, plus small amounts of structure 7 and structure 8 and starts melting at and decomposing at about 240° C.

### Formulations (Coatings) Used:

### Formulation 1:

Water-based clear base coat (without pigmentation): “Mischlack farblos ZW 42-6008-0101” (supplied by BASF Coatings GmbH). The solid content of the coating is 21.5%.

### Formulation 2:

Water-based white base concentrate: “Prüfweiβ ZU56-0PRW-0018” (supplied by BASF Coatings GmbH). The solid content of the coating is 52.0%.

### Formulation 3:

Water-based white base coat: 25 wt.-% “Mischlack ZW 42-6008-0101” and 75 wt.-% “Prüfweiβ ZU56-0PRW-0018”. The solid content of the mixed coating is 39.0%.

### Formulation 4:

Solvent-borne clear top coat (thermo-setting acrylic melamine) (The solid content of the coating is 53.0%.):

| | Weight-% |
|---|---|
| Viacryl ® SC 303 (60% in xylene/butanol; 26/9) | 30.2 |
| (acrylic resin, Allnex) | |
| Viacryl ® SC 370 (75% in SN/butylacetate) | 25.6 |
| (acrylic resin, Allnex) | |
| Maprenal ® MF 650 | 29.9 |
| (55% in isobutanol) (isobutylated melamine-formaldehyde | |
| resin, Ineos) | |
| Butyl acetate/butanol (37:8) (solvents) | 4.7 |
| Isobutanol (solvent) | 5.3 |
| Solvesso ® 150 (solvent, Exxon Mobil Chemicals) | 3.0 |
| Baysilone ® MA (1% in Solvesso 150) (leveling | 1.3 |
| agent, Momentive) | |
| | 100.0 |

### Inventive Example 1 (IE1)

1.5 wt. % UV absorber and 1.5 wt. % pigment 1, each based on the solid content, were added to formulation 2. The distribution of the pigment and the UV absorber (dispersing) was conducted by adding the same weight amount of 2 mm glass beads and shaking of the mixture for 2 hours using a Scandex shaker (manufacturer: Lau GmbH). Subsequently the glass beads were filtered off. The formulation was then mixed with formulation 1 in the weight ratio 3/1. The resulting mixture was applied (wire coater 50 μm) onto a glass plate resulting after cure (80°, 20 min) in a dry film thickness of 20 μm.

### Inventive Example 2 (IE2)

Inventive example 1 has been repeated except that 1.5 wt. % Pigment 2 based on the solid content was used instead of 1.5 wt. % pigment 1 based on the solid content.

### Reference Example 3 (RE3)

Inventive example 1 has been repeated except that no UV absorber and pigment have been used and the distribution step for 2 hours has been omitted.

The UV/vis transmittance has been determined on examples IE1, IE2 and RE3 as described above. The results are provided in the following table.

| | UV/VIS transmittance (% T) | | | | | |
|---|---|---|---|---|---|---|
| | 400 nm | 420 nm | 440 nm | 460 nm | 480 nm | 500 nm |
| RE3 | <0.1 | 5.1 | 7.4 | 8.2 | 8.9 | 9.6 |
| IE1 | <0.1 | <0.1 | <0.1 | <0.1 | 0.1 | 4.2 |
| IE2 | <0.1 | <0.1 | <0.1 | <0.1 | <0.1 | <0.1 |

As can be seen from the above the transmittance at 420 nm is already one order of magnitude lower compared with the reference. At higher wavelengths the transmittance of the reference increases, while the inventive examples show superior UV/Vis-protection up to 480 or 500 nm respectively.

### Photo-Stability

For determination of the photo stability a clear coat (formulation 4) was applied onto the above samples of IE1 and IE2 using a 125 μm wire coater (results in a dry film thickness of 40 μm after cure). In order to avoid cracking of the clear coat during exposure, the formulation did contain 1 wt. % Tinuvin 123 (based on resin solids). The layers were jointly cured at 130° C. for 30 min. The clear coat composition does not absorb in the wavelength range between 300 and 500 nm.

The UV/VIS spectrum of each sample was recorded and the specimens were subsequently subjected to the exposure conditions according to SAE J 2527. During exposure the UV/VIS spectra were recorded in intervals of 500 h. The changes in the UV/VIS absorption at the absorption edge (Δ % T) are given in the following table 2. The absorption edge is defined as the wavelength at which the transmittance is below 0.5%.

| | | Δ% T | | | |
|---|---|---|---|---|---|
| | Absorption edge [nm] | 500 h | 1000 h | 2000 h | 3000 h |
| IE1 | 480 | 0.1 | 0.1 | 0.1 | 0.1 |
| IE2 | 500 | 0 | <0.1 | <0.1 | n.a. |

By using only one pigmented layer and a clear coat on top (which itself is UV/VIS transparent) the pigmented layer is exposed to the maximum irradiation under the weathering conditions according to SAE J 2527.

As can be seen from table 2 the transmittance changes even after 3000 hours of accelerated weathering conditions are negligible.

### Migration Resistance

Aluminium substrate pre-coated with a white coil coating (polyester/melamine) was used as neutral substrate. The substrate was coated with IE1 or IE2 and dried (80° C., 20 min) resulting in a dry film thickness of 20 μm. Thereon a second layer using the formulation 3 without UV absorber and pigment (RE3) was applied and dried (80° C., 20 min) also resulting in a dry layer thickness of 20 μm. The yellowness was then determined by a photo-spectrometer and the b* value was calculated using the CIE-L*a*b* system.

| | b* value |
|---|---|
| IE1 | 1.7 |
| IE2 | 0.6 |

The low b* values show that the white color of the base coating is not strongly shifted to the yellowish area indication very low migration of the UV absorber and pigment into the second white base coat layer.

1. A coating composition, comprising
one or more compounds (A) according to the following formula (I) 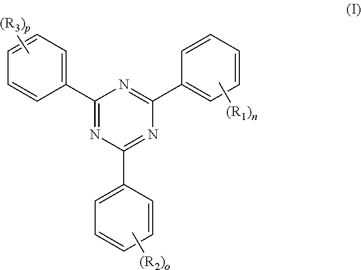
wherein
each R₁, R₂ and R₃ is independently
—OR₄, R₄ being independently hydrogen or a C₁ to C₁₅ alkyl group free from heteroatoms; or
C₁ to C₂₄ hydrocarbyl groups optionally containing heteroatoms;
n is 2, 3, 4 or 5;
o is 2, 3, 4 or 5;
p is 2, 3, 4 or 5;
with the proviso that at least one of R₁ and at least one of R₂ and at least one of R₃ is —OR₄
and
a pigment (B) which has a minimum integrated transmittance within the range of 380 to 600 nm.

2. The composition according to claim 1, wherein in component (A) at least two of R₁ and at least two of R₂ and at least two of R₃ are —OR₄.

3. The composition according to claim 1, wherein component (A) is a compound according to the following formula (II) 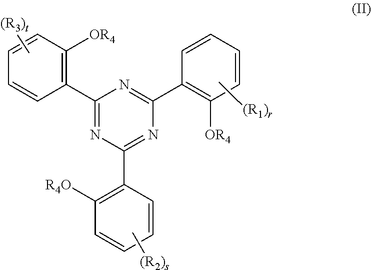
wherein
R₄ is hydrogen or a C₁ to C₁₅ alkyl group free from heteroatoms;
each R₁, R₂ and R₃ is independently
—OR₄, R₄ being hydrogen or a C₁ to C₁₅ alkyl group free from heteroatoms; or
C₁ to C₂₄ hydrocarbyl groups optionally containing heteroatoms;
r is 1, 2, 3 or 4;
s is 1, 2, 3 or 4; and
t is 1, 2, 3 or 4.

4. The composition according to claim 1, wherein the pigment (B) is selected from the group consisting of an isoindoline, an isoindolinone, a benzimidazolone, a quinophthalone, an azomethine Cu-complex, bismuth vanadate, a mixed metal oxide, an iron oxide, and a mixture thereof.

5. The composition according to claim 4, wherein the pigment (B) is an isoindoline or a quinophthalone.

6. The composition according to claim 1, wherein components (A) and/or (B) are each present in an amount of 0.1 to 30 wt. % based on a total solids content of the composition.

7. The composition according to claim 1, further comprising
one or more UV absorbers selected from the group consisting of a hydroxy-phenyl-benzotriaziole, a hydroxy-phenyl-triazine, a hydroxyl-benzophenone, an oxanilide, a cyanoacrylate, a malonate and a mixture thereof.

8. The composition according to claim 1, further comprising
a sterically hindered amine compound.

9. A coating, obtained by applying the composition according to claim 1 on a substrate.

10. The coating according to claim 9, whereby the coating is applied at a dry film thickness of 30 μm or less.

11. The coating according to claim 9, wherein the substrate is glass, metal, wood, a plastic or ceramic material or another coating layer applied on such a substrate.

12. A method for protecting a substrate against UV/Vis-radiation, the method comprising
applying a composition comprising
one more compound(s) (A) according to the following formula (I) 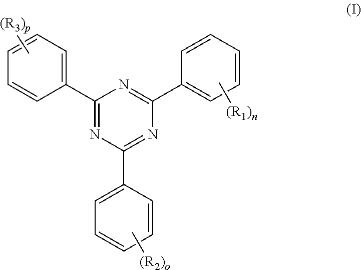
wherein
each R₁, R₂ and R₃ is independently
—OR₄, R₄ being hydrogen or a C₁ to C₁₅ alkyl group free from heteroatoms; or
C₁ to C₂₄ hydrocarbyl groups optionally containing heteroatoms;
n is 2, 3, 4 or 5;
o is 2, 3, 4 or 5;
p is 2, 3, 4 or 5;
with the proviso that at least one of R₁ and at least one of R₂ and at least one of R₃ is —OR₄
and
a pigment (B) which has a minimum integrated transmittance within the range of 380 to 600 nm.

13. The method according to claim 12, wherein the substrate is glass, metal, wood, a plastic or ceramic material or another coating layer applied on such a substrate.

14. A process for stabilizing a substrate against deleterious influence of UV/Vis-radiation, the process comprising
applying the coating composition according to claim 1 to the substrate.

15. The process of claim 14, wherein the coating composition is an automotive coating.

16. The process according to claim 14, wherein the substrate is glass, metal, wood, a plastic or ceramic material or another coating layer applied on such a substrate.
